(19) United States
(12) Patent Application Publication
(10) Pub. No.: US 2023/0078240 A1
(43) Pub. Date: Mar. 16, 2023

(54) INTERVENTIONAL UNMANNED OPERATION CHANMBER SYSTEM

(71) Applicant: BEIJING WEMED MEDICAL EQUIPMENT CO.,LTD., Beijing (CN)

(72) Inventor: Tao HUANG, Beijing (CN)

(73) Assignee: BEIJING WEMED MEDICAL EQUIPMENT CO.,LTD., Beijing (CN)

(21) Appl. No.: 17/989,712

(22) Filed: Nov. 18, 2022

Related U.S. Application Data
(63) Continuation of application No. PCT/CN2022/109592, filed on Aug. 2, 2022.

(51) Int. Cl. A61B 6/10; A61B 34/37; A61B 90/70; A61B 6/00; A61B 6/04; A61B 6/12

(52) U.S. Cl. CPC A61B 6/107; A61B 6/12; A61B 6/0407; A61B 6/481; A61B 6/0487; A61B 34/37; A61B 90/70; A61B 2034/301

(57) ABSTRACT

An interventional unmanned operation chamber system is provided. The system includes a catheter chamber, which is an area for interventional operation and is provided with a catheter bed therein. The control chamber is arranged close to the catheter chamber, and an observation window is arranged between the catheter chamber and the control chamber. The catheter chamber has intervenient operation robot, master control robot, puncture robot, catheter and guidewire replacing robot that cooperate with each other to work internally. A DSA device and a contrast agent injection device are arranged on the catheter bed. The monitoring device is arranged in the control chamber and is in communication with the robots, the DSA device and the contrast agent injection device, and is used for displaying information of each device and the robot, synchronously updating in real time and supervising by a doctor. The controller is arranged in the control chamber.

### CROSS-REFERENCE TO RELATED APPLICATIONS

The patent application is a continuation application of PCT/CN2022/109592, filed on Aug. 2, 2022. which claims the benefit and priority of Chinese Patent Application No. 202210858090.2. filed on Jul. 20, 2022, the disclosure of which is incorporated by reference herein in its entirety as part of the present application.

### TECHNICAL FIELD

The invention relates to the technical field of minimally invasive vascular interventional surgery, and more specifically, to an interventional unmanned operation chamber system.

### BACKGROUND

The minimally invasive interventional therapy of the cardiovascular and cerebrovascular diseases is a main treatment means aiming at the cardiovascular and cerebrovascular diseases. Compared with the traditional surgical operation, it has the obvious advantages of small incision, short postoperative recovery time and the like. The cardiovascular and cerebrovascular interventional operation is a process in which a doctor manually sends a catheter, a guide wire, a stent and other instruments into a patient to finish treatment.

The interventional surgery has the following problems: firstly, in the operation process, the DSA emits X-rays, the physical strength of a doctor is reduced quickly, the attention and the stability are also reduced, the operation precision is reduced, accidents such as endangium injury, perforation and rupture of blood vessels and the like caused by improper pushing force are easy to occur, and the life risk of a patient is caused. Second, the cumulative damage of long-term ionizing radiation can greatly increase the probability of doctors suffering from leukemia, cancer and acute cataract. The phenomenon that doctors accumulate rays continuously because of interventional operation becomes a problem that the occupational lives of the doctors are damaged and the development of the interventional operation is restricted to be neglected.

With the robot technologies, the robot can complete the operation process of the interventional operation without manual participation, and the problems are effectively solved. Obviously, it is necessary to monitor the operation manually during the robotic surgery. The whole interventional operation process is completed by the mutual cooperation of various robots, and the establishment of an interventional unmanned operation room is a future development trend.

However, the current intervention operation has the following problems: (1) In interventional surgerys, all procedures require hands-on from a doctor, the doctor is under heavy pressure and the task is heavy, from the puncture process, the intraoperative control of a catheter guide wire, the transport to a patient, the delivery of consumables, the injection of contrast media, the replacement of surgical instruments, the analysis of images and the like; (2) The doctor wears the lead coat for a long time and has serious damage to the skeleton and the muscle of the body; (3) Doctors receive the damage of DSA radiation for a long time and have serious influence on the health; (4) the use of the catheter chamber is frequent, and the cleaning is often problematic; (5) Transport to patients often requires more human resources; (6) The manual control precision of the guide wire of the catheter is not high, the operation efficiency is not high, and the like.

Therefore, how to provide an interventional unmanned operation chamber system is a problem that needs to be solved by those skilled in the art.

### SUMMARY

The disclosure aims at solving, at least to some extent, one of the above-mentioned problems in the prior art.

To this end, the object of the disclosure is to propose an interventional unmanned operation chamber system, solving above mentioned problems 1-3 and 6.

An interventional unmanned operation chamber system is provided which includes a catheter chamber, the catheter chamber being an area of interventional surgery having a catheter bed therein; a control chamber, the control chamber is arranged next to the catheter chamber, and an observation window is arranged between the catheter chamber and the control chamber; the robot, the pipe room has intervenient surgical robot, master control robot, puncture robot, catheter and guidewire replacing robot that cooperate with each other; a DSA device and a contrast agent injection device are arranged on the catheter bed; the monitoring device is arranged in the control chamber and is in communication with the robot, the DSA device and the contrast agent injection device, and is used for displaying information of each device and the robot, updating in real time and synchronously and supervising a doctor; and the controller is arranged in the control chamber and used for man-machine interaction between the doctor and the robot.

According to the technical scheme, compared with the prior art, the invention discloses an interventional unmanned operation chamber system, a catheter chamber and a control chamber are designed in a close-proximity mode, a plurality of robots with different functions and working in a matched mode are arranged in the catheter chamber, a DSA device and a contrast agent injection device are arranged on a catheter bed and are matched to complete an operation, the operation at least comprises the operations of image diagnosis, operation puncture, catheter guide wire insertion, catheter guide wire replacement, catheter guide wire movement, angiography and the like, the operation precision is improved, a monitoring device for monitoring various state information and a human-computer interaction controller between a doctor and a robot are arranged in the control chamber, so that the purpose of unmanned interventional operation is achieved through the combination of the robots, the problems of high pressure and heavy task of an interventional operator are solved, the doctor does not need to wear a lead garment in the control chamber for a long time, meanwhile, the damage of receiving DSA radiation for a long time is avoided, and the influence of the interventional operation on the health of the doctor is reduced.

Further, a ward is provided adjacent to the catheter chamber for resting the patient.

Further, an automatic transfer trolley is moved between the ward and the catheter chamber and used for automatically transferring the patient. The problem 5 is solved, and more human resources are not needed for the transportation of the patient.

Furthermore, a transfer trolley charging pile is fixed in the ward and used for charging the automatic transfer trolley.

Further, the robot further comprises a consumable delivery robot that records surgical consumable information for delivering surgical consumables to the catheter chamber, which is communicatively connected to the monitoring device and the controller.

Further, the robot still includes fast charging robot, fast charging robot and a plurality of robot communication connection for change the battery that the electric quantity is low.

Furthermore, a charging area for charging a battery with low electric quantity is arranged outside the catheter chamber, and a charging position for the fast charging robot is arranged in the charging area.

The cleaning robot is in communication with the controller and is used for automatically cleaning the catheter chamber after an operation is finished. Problem 4 is solved, need not the frequent clean pipe room of manpower, improves clean efficiency.

Further, the monitoring device comprises a plurality of display screens supported by the screen support.

Further, the authority of the robot has priority, the master control robot has the maximum authority, and the master control robot is an operation instructor and is used for image diagnosis and instructing other robots to work cooperatively.

### BRIEF DESCRIPTION OF DRAWINGS

In order to more clearly illustrate the embodiments of the disclosure or the technical solutions in the prior art, the drawings used in the description of the embodiments or the prior art will be briefly described below, it is obvious that the drawings in the following description are only embodiments of the disclosure, and for those skilled in the art, other drawings can be obtained according to the provided drawings without creative efforts.

FIG. 1 illustrates an overall layout of the interventional unmanned operation chamber system.

FIG. 2 illustrates a layout of an interventional unmanned operation chamber system within a catheter chamber.

FIG. 3 illustrates a flow diagram of an interventional unmanned operation chamber system.

FIG. 4 is a schematic diagram of a master control robot.

FIG. 5 illustrates a schematic structural diagram of a finger device of a master control robot.

FIG. 6 illustrates an exploded view of a finger device of a master control robot.

FIG. 7 is a schematic diagram of the puncture robot.

FIG. 8 is a schematic diagram of the first finger device of the puncture robot.

FIG. 9 is a schematic view of the second finger device of the puncture robot.

FIG. 10 is a schematic diagram of a catheter and guidewire replacing robot.

FIG. 11 is a schematic structural diagram of an arm component and a head of the catheter and guidewire replacing robot.

FIG. 12 is a top view of the arm component of the catheter and guidewire replacing robot.

FIG. 13 is a schematic view of a replacement guidewire catheter.

FIG. 14 is a schematic structural diagram of the fast charging robot.

FIG. 15 is a schematic structural view of a clamping and pushing mechanism of the fast charging robot.

FIG. 16 is a structural diagram of a consumable delivery robot.

FIG. 17 is a schematic structural view of an arm assembly of the consumable delivery robot.

FIG. 18 is a schematic view of a cleaning robot.

FIG. 19 is a schematic structural diagram of a base device of the cleaning robot.

FIG. 20 is a schematic view showing the structure of the automatic transfer trolley.

FIG. 21 is a schematic view showing the bottom structure of the automatic transfer trolley.

FIG. 22 is a schematic structural view of the automatic charging and fixing device of the automatic transfer trolley.

FIG. 23 is a schematic view of a monitoring device.

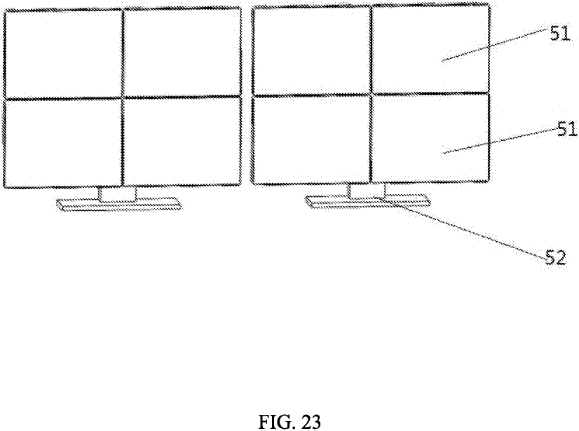

The main reference numbers are as follows:

catheter chamber 1, catheter bed 101, control chamber 2, observation window 201. DSA device 3, contrast agent injection device 4. monitoring device 5, display screen 51, screen support 52, controller 6. ward 7, automatic transfer trolley 8, transfer trolley charging post 81. charging area 9, interventional surgery robot Q1, master control robot Q2, puncture robot Q3, catheter and guidewire replacing robot Q4, consumable delivery robot Q5, fast charging robot Q6, cleaning robot Q7.

### DETAILED DESCRIPTION OF THE EMBODIMENTS

In order to make the technical solutions of the disclosure better understood, the disclosure is described in detail below with reference to the accompanying drawings and the detailed description. The embodiments of the disclosure will be described in further detail below with reference to the drawings and specific embodiments, but the disclosure is not limited thereto.

Reference will now be made in detail to embodiments of the disclosure, examples of which are illustrated in the accompanying drawings, wherein like or similar reference numerals refer to the same or similar elements or elements having the same or similar function throughout. The embodiments described below with reference to the drawings are illustrative and intended to be illustrative of the invention and are not to be construed as limiting the invention.

In the description of the disclosure, it is to be understood that the terms "upper", "lower", "front", "rear", "left", "right", "vertical", "horizontal", "top", "bottom", "inner", "outer", etc., indicate orientations or positional relationships based on those shown in the drawings, and are for convenience of description and simplicity of description only, but do not indicate or imply that the referenced devices or elements must have a particular orientation, be constructed and operated in a particular orientation, and thus, are not to be construed as limiting the disclosure.

Furthermore, the terms “first”, “second” and “first” are used for descriptive purposes only and are not to be construed as indicating or implying relative importance or to implicitly indicate the number of technical features indicated. Thus, a feature defined as “first” or “second” may explicitly or implicitly include one or more of that feature. In the description of the disclosure, “a plurality” means two or more unless specifically defined otherwise.

In the disclosure, unless otherwise explicitly stated or limited, the terms “mounted.” “connected,” “fixed,” and the like are to be construed broadly, e.g.. as being permanently connected, detachably connected, or integral; can be mechanically or electrically connected; either directly or indirectly through intervening media, either internally or in any other relationship. The specific meanings of the above terms in the disclosure can be understood according to specific situations by those of ordinary skill in the art.

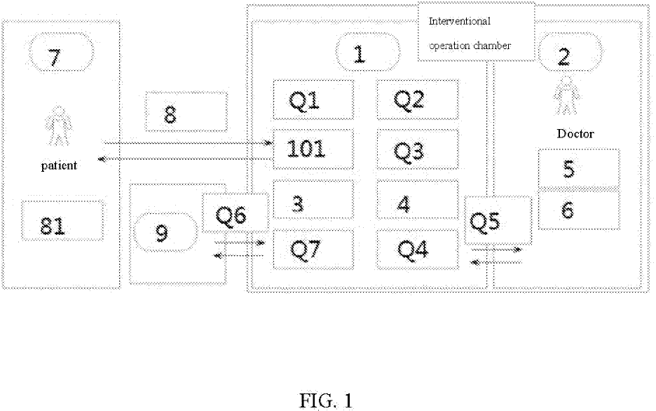

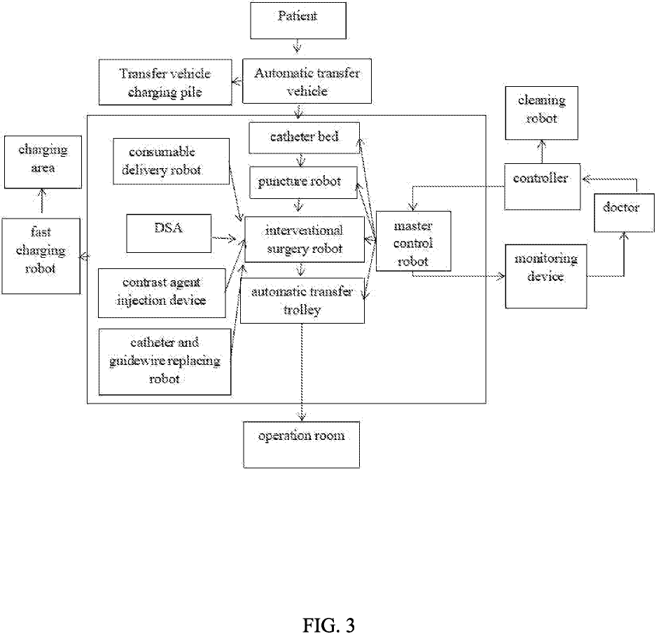

Multiple problems for the existing interventional operation have been indicated in the background of the disclosure. An interventional unmanned operation chamber system is provided in the embodiments, see for example FIGS. 1-3. The system includes a catheter chamber 1, a control chamber 2, monitoring device 5 and a controller 6. The catheter chamber 1 is an interventional surgery area and is internally provided with a catheter bed 101.

The control chamber 2 is arranged next to the catheter chamber 1, and an observation window 201 is arranged between the catheter chamber 1 and the control chamber 2.

An interventional surgery robot Q1, a master control robot Q2, a puncture robot Q3 and a catheter and guidewire replacing robot Q4 which are matched with each other are arranged in the catheter chamber 1. A DSA device 3 and a contrast agent injection device 4 are arranged on the catheter bed 101.

The monitoring device 5 is arranged in the control chamber 2, and is in communication with the robot. The DSA device 3 and the contrast agent injection device 4, is used for displaying information of each device and the robot, and is used for real-time synchronous updating and monitoring by doctors.

The controller 6 is arranged in the control chamber 2 and used for man-machine interaction between a doctor and the robot

The invention discloses an interventional unmanned operation chamber system, wherein a catheter chamber and a control chamber are designed to be adjacent to each other, and a number of robots with different functions and working in a matched mode are arranged in the catheter chamber, a DSA device and a contrast agent injection device are arranged on a catheter bed, and the operation is completed in a matched mode. The operations at least include the operations of image diagnosis, operation puncture, catheter guide wire placement, catheter guide wire replacement, catheter guide wire movement, angiography and the like. The operation precision is improved, a monitoring device for monitoring information of various states and a human-computer interaction controller between a doctor and the robots are arranged in the control chamber, so that manual monitoring is realized, the aim of the unmanned interventional operation is realized by the combination of the robots. The problems of high pressure and heavy task of an interventional operator are solved. The doctor does not need to wear a lead garment in the control chamber for a long time, meanwhile, the injury of receiving DSA radiation for a long time is avoided, and the influence of the interventional operation on the health of the doctor is reduced.

In one embodiment of the invention, ward 7 is provided adjacent to the catheter chamber 2 for the resting of the patient. Preferably the ward is arranged opposite to the catheter chamber.

In one embodiment of the invention, an automatic transfer trolley 8 is moved between the ward 7 and the catheter chamber 1 for automatically transferring the patient, thereby eliminating the need to expend significant manpower in transporting the patient.

Advantageously, a transfer trolley charging post 81 is fixed in the ward 7 for charging the automatic transfer trolley 8.

In one embodiment of the invention, the robot further includes a consumable delivery robot Q5. The consumable delivery robot Q5 records surgical consumable information for delivering surgical consumables to the catheter chamber 1, which communicates with the monitoring device 5 and the controller 6.

In one embodiment of the invention, the robot further includes a fast charging robot Q6. The fast charging robot Q6 is in communication with a plurality of robots and is used for replacing batteries with low electric quantity. Therefore, the problem that the robot cannot timely supplement the battery with low electric quantity is solved.

Advantageously, a charging area 9 for charging the battery with low capacity is arranged outside the catheter chamber 1, and a charging position for the fast charging robot Q6 is arranged in the charging area 9.

In other embodiments of the disclosure, the cleaning robot Q7 is communicatively connected to the controller 6, and the cleaning robot Q7 is used for automatically cleaning the inside of the catheter chamber 1 after the operation is finished. Therefore, the catheter chamber does not need to be cleaned frequently by manpower, and the cleaning efficiency is improved.

Advantageously, the monitoring device 5 includes a number of display screens 51, which are each supported by the screen support 52.

In the above embodiments, the authority of the robot has priorities, and the master control robot Q2 has the maximum authority, and is an operator for performing an operation, which is used for image diagnosis and instructing other robots to work in cooperation.

In one embodiment of die disclosure, the layout of the interventional unmanned operating chamber can be divided into four spaces. In the ward, an electric charging pile for the automatic transfer trolley is installed. In the charging area, a charging device is provided. The catheter chamber includes an interventional surgery robot, a catheter bed, a DSA device, a cleaning robot, a master control robot, a puncture robot, a contrast agent injection device, a catheter and guidewire replacing robot. The control chamber includes a monitoring device and a controller. Several robots function in different areas. They are each automatic transfer trolley that move between wards and catheter chambers. The fast charging robot moves between a charging area and a catheter chamber. A consumable delivery robot moves between the catheter chamber and the control chamber.

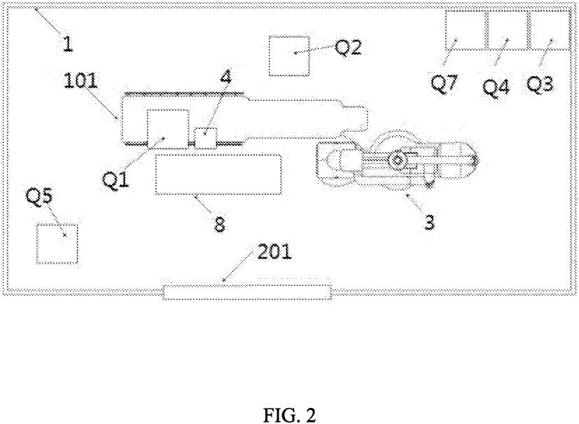

Referring to FIG. 2, in the catheter chamber 1, a observation window 201 is made of lead glass, which facilitates the control chamber for viewing the catheter chamber. The guide pipe machine 101 is installed at the center of the catheter chamber 1. The DSA device 3 is installed at the head of the guide pipe machine 101, and the master control robot Q2 moves on the left side of the guide pipe machine 101. After the automatic transfer trolley 8 enters the catheter chamber, the right side of the catheter bed 101 is stopped, and after the patient gets on the catheter bed 101, the automatic transfer trolley 8 exits the catheter chamber and waits at the doorway. The interventional surgery robot Q1 is mounted on a rail on the side of the catheter bed 101. The contrast injection device 4 is placed on the catheter bed 101. The cleaning robot Q7, the catheter and guidewire replacing robot Q4 and the puncture robot Q3 are respectively parked in the corners of the catheter chamber. When needed, they can be moved out for work. The consumable delivery robot Q5, after taking out the consumable, is placed on the catheter bed 101, and then exits the outside of the catheter chamber for standby.

The complete procedure for the entire interventional surgery is roughly as follows: first, the patient waits in the ward and, after receiving a message that the surgery can be performed, the automatic transfer trolley moves freely. The patient lies on the automatic transfer trolley, and the automatic transfer trolley automatically transports the patient to the catheter chamber along a preset route according to the GPS guidance and stops at the side of the catheter bed. Then after the patient moves from the transfer trolley to the catheter bed, the transfer trolley automatically exits the catheter chamber, stays outside the catheter chamber and standby for the end of the operation. After the patient lies, the master control robot instructs the puncture robot to start to act, the puncture robot can move to the side face of the catheter bed, and the actions of disinfecting, local anesthesia, puncturing, implanting the outer sheath and the like to the patient step by step are started. After the finish of each performance, the message is transmitted to the master control robot. The master control robot starts to instruct the DSA device and the interventional surgery robot to start for operation. Under the assistance of the master control robot, the interventional robot is matched with the image of the DSA device to sequentially complete the placement of the contrast guide wire and the contrast catheter at the proper position of the blood vessel of the patient, the master control robot indicates the contrast agent injection device to inject the contrast agent, and the image of the blood vessel can be displayed on the DSA device. The angle of the DSA is adjusted, and imaging of different positions is carried out, so that more comprehensive blood vessel morphology can be observed. After different blood vessel images are obtained, the master control robot can perform disease state analysis according to the blood vessel images, and a diagnosis result is obtained after the master control robot compares and analyzes the blood vessel images with big data. If the operation needs to be continued, the DSA device and the interventional robot cooperate to carry out the placement of the guide catheter. Meanwhile, the master control robot instructs the catheter and guidewire replacing robot to assist in completing the replacement of the guidewire and catheter. Simultaneously, master control robot can select the required consumptive material of operation, sends the consumptive material and delivers the robot, or sends the instruction through the controller, and the consumptive material delivers the robot and seeks, and after finding the target consumptive material, the consumptive material delivers the robot and can place the operation consumptive material on the pipe lathe, and master control robot can assist the completion and install the consumptive material on interveneeing the operation robot. The pushing of the surgical consumables (e.g., stent) is then done in steps, based on the DSA images, during which multiple fits of the contrast injection device are required. The master control robot can complete the control of DSA pedal during operation, and after one-step operation, the operation treatment is completed smoothly.

Then, with the aid of the master control robot, all guide wire catheters are withdrawn from the body. The master control robot helps the patient to complete the dressing. A message is sent to the automated transfer vehicle to enter the catheterization room to stop at the side of the catheterization bed, the patient returns from the catheterization bed to the transfer vehicle, and the transfer vehicle returns to the ward with the patient and stops in place.

During the operation, the states and data of all the devices are on the monitoring device in the control chamber, so that the doctor can supervise the operation in the whole operations. Once any abnormality is found, the operation can be suspended and adjusted at any time through the controller. After the abnormality is fixed, the operation can continue. The doctor may also use the controller to control the robot. After one operation is finished, the master control robot instructs the cleaning robot to clean the catheter chamber, or the controller sends a cleaning robot cleaning command to prepare for the next operation.

Each robot has all adopted lithium battery powered, if it is detected that certain robot has low electric quantity, the fast charging robot is signaled to carry a lithium cell that is full of the electricity and remove and change for the robot next door to the robot to take the battery of low electric quantity back to fill and charge on the electric pile.

The robot and the device applied in the above operation flow may be a robot capable of realizing corresponding functions, and the following robot may be adopted as an individual robot, and the functions of the robot are described only by an individual example.

The interventional surgery robot Q1 can be a general interventional radiography and therapeutic operation robot disclosed in patent document CN112353491A, and is connected to a catheter bed. The master control robot Q2 can move in the catheter chamber by adopting a vehicle body structure. The robot monitors all parts including DSA, a catheter bed, other robots and the like during operation, can master the operation flow through machine learning, sends instructions to guide all equipment to perform matching actions, and sends various data to a monitoring system in real time. After the operation is finished, the robot can move to the corner by itself, and other operations are not influenced.

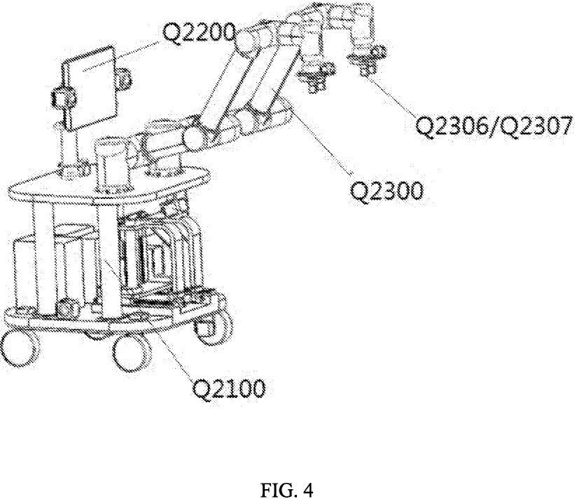

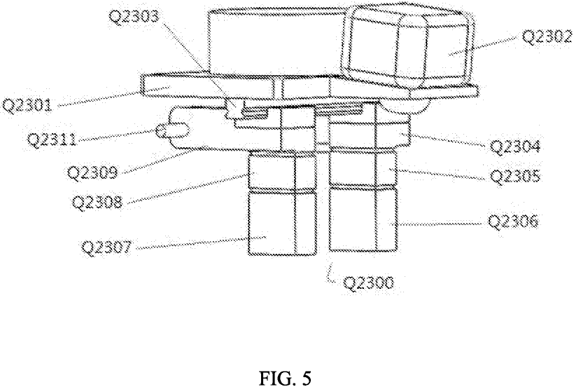

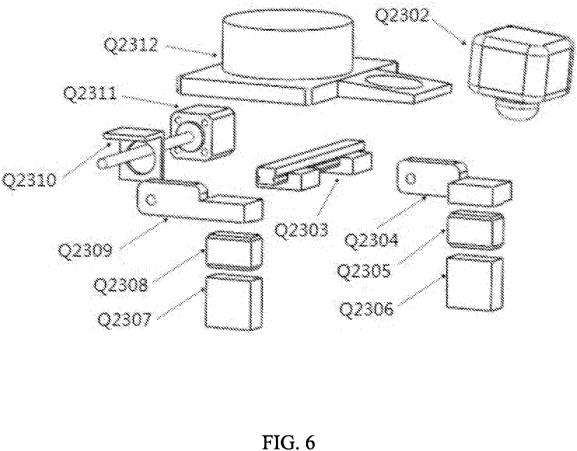

Referring to FIGS. 4-6, the master control robot Q2 includes a base device Q2100, head and arm component Q2200 and finger device Q2300. The base device Q2100 is provided with a walking device at the bottom for moving to a target area, and a pedal device for simulating the foot of a doctor to control the perspective and exposure action of the DSA is arranged on the walking device. The head and arm component Q2200 is supported on the top of the base device Q2100 and is used for completing identification information and positioning fingers. The finger device Q2300 is connected to the arm end of the head and arm component Q2200 and is used for clamping, replacing and installing surgical equipment.

The finger device Q2300 includes the finger connecting plate Q2301. a finger guide rail Q2303, a finger lead screw motor Q2311, connecting piece Q2304, Q2309 and two fingers (Q2307 and Q2306) perform relative movement or opposite movement. The finger connecting plate Q2301 is connected with the end part of the arm. The bottom of the finger connecting plate Q2301 is connected with a finger guide rail Q2303, and two finger sliding blocks slide on the finger guide rail Q2303. A finger lead screw motor Q2311 is fixed below the finger connecting plate Q2301 through a finger motor bracket Q2310. Each finger sliding block is correspondingly connected with one connecting piece Q2304, Q2309, and the two connecting pieces Q2304, Q2309 are respectively provided with clockwise threads and anticlockwise threads which are in threaded fit with the finger lead screw motor Q2311. When the finger lead screw motor Q2311 rotates. Each connecting piece Q2304 and Q2309 are correspondingly connected with one of the three-dimensional pressure sensors Q2305 and Q2308. The fingers Q2306 and Q2307 are connected to the lower portion of each three-dimensional pressure sensor Q2305 and Q2308, the inner surfaces of the fingers Q2306 and Q2307 are made of soft medical silica gel, and surgical equipment can be prevented from being damaged.

The three-dimensional pressure sensor arranged in the finger device can sense the clamping force and guarantee the clamping accuracy.

Advantageously, the finger connecting plate Q2301 extends obliquely outward to form a finger camera holder, and at least one finger camera Q2302 is connected to the finger camera holder, and the finger camera 302 is disposed toward the fingers Q2306 and Q2307.

After the object is clamped, the three-dimensional pressure sensors Q2305 and Q2308 can sense pressure values, and once a certain value is reached, the finger lead screw motor Q2311 stops moving. In the clamping process, the finger camera Q2302 pays attention to the shape of the object at any time so as to ensure that the situation of clamping the damaged equipment cannot be sent. Finger camera Q2302 is used for observing the environment of finger tip, can make things convenient for the distribution and the accurate position of the better observation object of robot and doctor.

It is worth to be noted that, before the operation starts, the master control robot Q2 will automatically move to the side of the catheter bed to supervise and guide the operation of each device, and take charge of the actions of clamping and replacing the operation equipment in the operation. After the operation is finished, the robot can automatically move into the corner, and the use and the cleaning of other equipment are not influenced. Before the robot is used for the first time, learning and training are needed, the purpose is to adapt the robot to the environment of a catheter chamber and to be familiar with equipment such as DSA a catheter bed and other robots used in the current medical application, and the learning and other equipment can be well matched to operate. After times of training, the robot can gradually master and memorize the learned knowledge, and after complete learning, the robot can be normally used for automatic surgery or auxiliary surgery. The robot system stores a large amount of operation images and standard operation flows and a plurality of abnormal condition processing measures, and can be used in actual clinic. The robot has an autonomous learning function, and can record and analyze the situation of each subsequent operation so as to select an optimal solution in the subsequent operation.

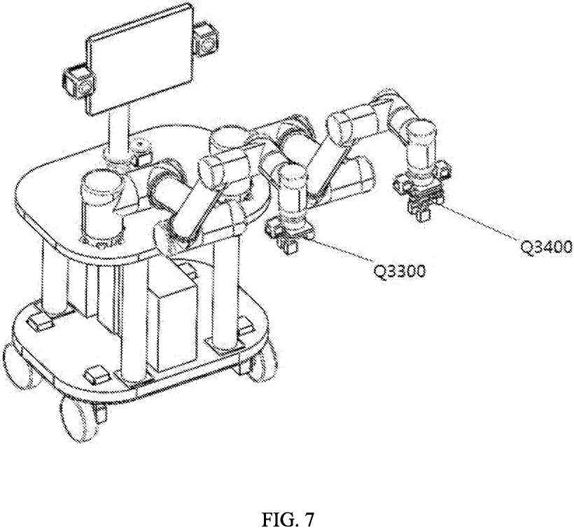

The puncture robot Q3, referring to FIG. 7, includes a base device, head and arm components, and puncturing finger devices, which are respectively connected to a first finger device Q3300 and a second finger device Q3400 through two mechanical arms. In the base device, information is received, stored, information is processed and sent through a host machine of the puncture robot. A first finger device is used for position a puncturing point of puncturing operation, and a second finger device is used for puncturing. During operation, the two cooperate with each other to grasp, replace and install the surgical equipment, which realizes the robotic puncture operation, improves the accuracy of puncture, and further reduces repeated operation due to inaccurate puncture position. It can make the condition of vasospasm happen and improve the safety of puncture operation.

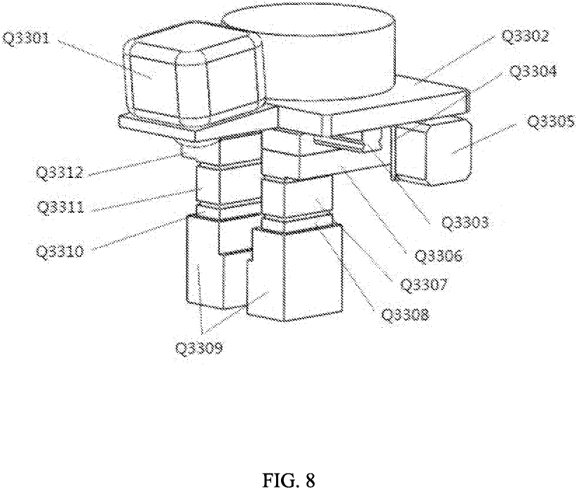

Specifically, referring to FIG. 8, the first finger device Q3300 includes a first finger connecting plate Q3302, a first finger guide rail Q3303, first finger lead screw motor Q3305, first connecting plate Q3306, Q3312, a first three-dimensional pressure sensor Q3307. Q3311 and a first finger consumptive material Q3309. The first finger connecting plate Q3302 is connected with the end part of a mechanical arm. The bottom of the first finger connecting plate Q3302 is connected with the first finger guide rail Q3303, and two first finger sliding blocks are slid on the first finger guide rail Q3303. The first finger lead screw motor Q3305 is fixed below the first finger connecting plate Q3302 through a first finger motor bracket Q3304. Each of the first finger sliding blocks is correspondingly connected with one first connecting plate Q3306. Q3312, and the two first connecting plates Q3306, Q3312 are respectively provided with clockwise threads and anticlockwise threads which are matched with the first finger lead screw motor Q3305 in a threaded manner. One of the first three-dimensional pressure sensors Q3307, Q3311 is connected to a lower portion of each of the first connecting pieces Q3306. Q3312. Each first three-dimensional pressure sensor Q3307. Q3311 below is connected with the first finger consumptive material Q3309. An internal surface of the first finger consumptive material Q3309 is made of soft medical silica gel.

Advantageously, a first electromagnet Q3308, Q3310 is fixed between the first three-dimensional pressure sensor Q3307, Q3311 and the first finger consumable Q3309, and the first finger consumable Q3309 has a first iron sheet magnetically connected to the first electromagnet Q3308. Q3310 inside.

A recess is formed inside the first finger consumptive material Q3309, and the first iron sheet is put into in the recess and is connected with the electromagnet magnetism, and the convenience is to the change of consumptive material. The first finger consumable Q3309 is a disposable consumable sterilized with ethylene oxide, and a new set is used for each operation. The inner surface of the first finger consumable Q3309 is made of soft medical silica gel, so that slipping and damage to surgical equipment can be prevented.

More advantageously, a side of the first finger connecting plate Q3302 extends obliquely outward to form a first finger camera support. At least one first finger camera Q3301 is connected to the finger camera support, and the first finger camera Q3301 is arranged towards the first finger consumable Q3309 direction. The first finger camera Q3301 is used to observe the environment of the finger tip.

One of the first connection pieces Q3306, Q3312 is clockwise threaded and the other of the first connection pieces Q3306, Q3312 is counterclockwise threaded, so that when the motor is rotated, the two first finger consumables Q3309 move relatively or oppositely to clamp or withdraw the object. After the object is clamped, the first three-dimensional pressure sensors Q3307, Q3311 sense the pressure value, and after a certain value is reached, the motor stops moving. In the clamping process, the first finger camera Q3301 pays attention to the shape of the object at any time to ensure that no broken equipment is sent.

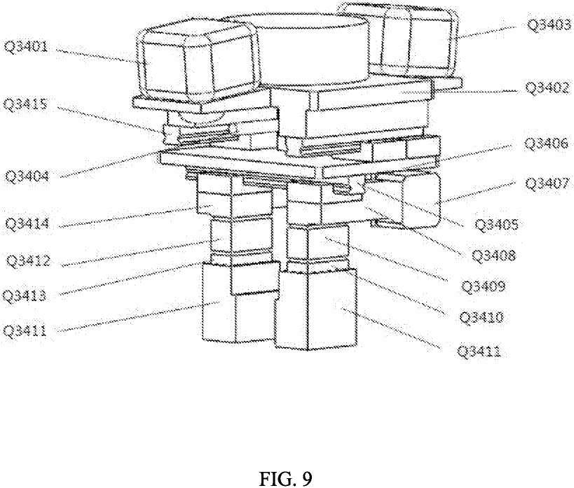

Referring to FIG. 9, the second finger device Q3400 includes a second finger connecting plate Q3402, two second finger guide rails Q3415. a second finger screw motor Q3404, two third finger guide rails Q3405 and a transition plate Q3406. The top of the second finger connecting plate Q3402 is connected with the end part of another mechanical arm, and the bottom of the second finger connecting plate Q3402 is formed with two mounting strips which are arranged in parallel and protrude downwards. The second finger guide rails Q3415 are correspondingly installed below the mounting strips. At least two second finger sliding blocks are slided on each second finger guide rail Q3415. The second finger screw motor Q3404 is fixed below the second finger connecting plate Q3402 through a second finger motor bracket and is positioned between the two mounting strips. The top of the transition plate Q3406 is provided with a connecting block in threaded connection with a lead screw of the second finger lead screw motor Q3404, and the transition plate Q3406 is fixed on the bottom surface of the second finger slide block. Two third finger guide rails Q3405 are arranged at the bottom of the transition plate Q3406 in parallel, and are arranged perpendicular to the second finger guide rails Q3415, and at least two third finger sliding blocks slide on the lower part of each third finger guide rail Q3405. The third finger lead screw motor Q3407 is fixed between the two second finger guide rails Q3415 through a motor bracket. Each third finger sliding blocks is correspondingly connected with one second connecting piece Q3408, Q3414, and the two second connecting pieces Q3408, Q3414 are respectively provided with clockwise threads and anticlockwise threads matched with the screw threads of the third finger screw motor Q3407. One second three-dimensional pressure sensor Q3409, Q3412 is correspondingly connected below each second connecting piece Q3408, Q3414. One second finger consumable Q3411 is connected to the lower part of each second three-dimensional pressure sensor Q3409, Q3412, and the inner surface of the second finger consumable Q341 1 is made of soft medical silica gel.

A recess is formed inside of the second finger consumptive material Q3411. The second iron sheet is put into in the recess and is connected with the electromagnet magnetism, which is convenient for the change of the consumptive material. The second finger consumable Q3411 is a disposable consumable sterilized with ethylene oxide, and a new set is used for each operation. The inner surface of the second finger consumable Q3411 is made of soft medical silica gel, so that slipping and damage to surgical equipment can be prevented.

Advantageously, a second electromagnet Q3410, Q3413 is fixed between the second three-dimensional pressure sensor Q3409, Q3412 and the second finger consumable Q3411, and a second iron piece magnetically connected with the second electromagnet Q3410, Q3413 is arranged in the second finger consumable Q3411.

More advantageously, the second finger connecting plates Q3402 extend obliquely towards two sides to form second finger camera supports, each second finger camera support is connected with at least one second finger camera Q3401, Q3403. and the second finger cameras Q3401, Q3403 are arranged towards the direction of the second finger consumable Q3411.

One second connecting piece Q3408, Q3414 are clockwise threads, and the other second connecting piece Q3408, Q3414 are anticlockwise threads, so that when the third finger screw motor Q3407 (the third herein means the sequence of the screw motors, not the third finger) rotates, the two second finger consumables Q3411 carry out relative motion or opposite motion, and the object is clamped and withdrawn. After the object is clamped, the second three-dimensional pressure sensors Q3409 and Q3412 sense the pressure value, and once a certain value is reached, the motor stops moving. During the clamping, the second finger cameras Q3401 and Q3403 pay attention to the shape of the object at any time so as to ensure that the condition of clamping the damaged equipment is not sent. The whole finger device below is driven to move left and right under the drive of the second finger screw motor Q3404, and the reciprocating propelling action of a guide wire, an outer sheath and the like can be realized by matching with the clamping action of the third finger screw motor Q3407. Specifically, the second finger screw motor Q3404 rotates to enable the second finger consumable Q3411 to move to the rightmost end, the third finger screw motor Q3407 rotates to enable the second finger consumable Q3411 to clamp a guide wire or a sheath, the second three-dimensional pressure sensors Q3409 and Q3412 are used for sensing clamping force. After clamping, the second finger screw motor Q3404 is rotated to enable the finger to move to the leftmost end, the third finger screw motor Q3407 opens the guide wire or the sheath, the second finger screw motor Q3404 is rotated to return to the original position, and the operation is repeated until the required position is reached.

During the puncturing of the surgical puncture robot, the radial artery puncture of the wrist is taken as an example for explanation. Once the operation is started, the required puncture operation consumables are placed on the catheter bed, and the robot moves to the side of the catheter bed. The first finger device of the robot is used to find the wrist of the patient, and after positioning to the position of the radial artery, the finger can be used to feel the pulse of the radial artery. One finger can be used to feel the pulse while the other finger is suspended. The three-dimensional pressure sensor on the felt finger consumables searches for the pulse, if the position is not right during the search, the position is changed and the search is continued. If the pulse is felt, the finger camera will locate the position and this position functions as the puncture point During the pulse detection, if the pressure value is a regularly changing numerical value, it is indicate as a pulse; if the pressure value is unchanged, it is not indicated as a pulse, and the three-dimensional pressure sensor can be FA702-D, or silicon piezoresistive type. The volume of the pressure sensor can be selected according to the use. The robot’s first finger device grabs a cotton ball dipped in alcohol and wipes the skin of the radial artery appendage. The robotic first finger device grasps the syringe of anesthetic agent, moves to the radial artery appendage, and gently penetrates the skin. The second finger device of the robot pushes a dose of the syringe and stops it The first finger device holds the syringe. After waiting for a moment, the first finger device of the robot is used to gently grasp the arm of the patient, the second finger device of the robot is used to grasp the puncture needle, and the second finger camera is used to find the puncture point. Driven by the second finger lead screw motor, the puncture needle is slowly put into the skin, and at the same time, two cameras of the second finger device observe whether there is blood returning phenomenon, and stop moving the motor when there is blood returning. The first finger device of the robot moves slowly to grasp the puncture needle and the second finger device of the robot pulls out the needle core. The first finger device is lowered a little slowly, and then the second finger device grabs the puncture guide wire and passes it into the puncture needle. The guide wire is slowly pushed back and forth for a certain distance. The first finger device is held in place of the puncture opening and the second finger device is pulled out of the puncture needle tube. The second finger device grasps the outer sheath and the first finger device grasps the end of the piercing guide wire. The second finger device threads the outer sheath into the guidewire, then the first finger device holds the puncture site, and the second finger device threads the outer sheath into the vessel along the guidewire, pushes the outer sheath forward until the outer sheath reaches the site of the puncture site until the whole puncture process is completed. After the puncture is completed, the robot returns to the corner without affecting the follow-up of the surgery.

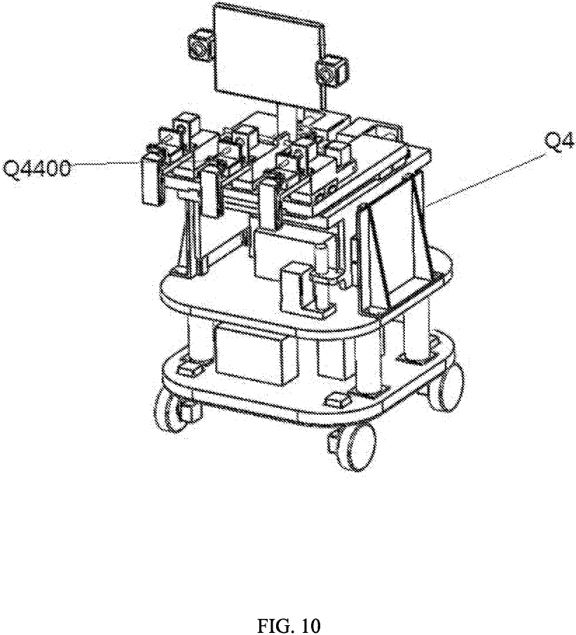

The catheter and guide wire replacing robot Q4 is used in interventional operations, and replacement operation is carried out on interventional operation consumables such as guide wires, balloons or stent catheters and the like, referring to the attached FIGS. 10-13. The guide wire catheter can be replaced by matching with an interventional surgery robot, and can also be replaced by matching with the assistance of doctors. The invention is used for completing the operation actions of threading and withdrawing of a guide wire, threading a catheter into the guide wire and pushing the catheter into a Y valve, withdrawing the catheter from the guide wire and the like, which ensures that the catheter and the guide wire do not displace in the process of replacing the guide wire catheter, thereby ensuring the safety of the operation.

The catheter and guidewire replacing robot Q4 includes a base device, a head and a driving arm component Q4400. The driving arm component Q4400 is used for completing the action of replacing the guide wire catheter. Three groups of arms are arranged, and each arm functions independently. The three groups of arms are arranged on a connecting plate, and the connecting plate can move back and forth through the two groups of screw rod motors and the two groups of linear guide rails and is used for extending and retracting the handle arms. The three groups of arms have basically the same structure and can move left and right. Each set of arms has a clamping mechanism for clamping and withdrawing a guide wire or catheter. A pressure sensor is arranged in the clamping device and used for detecting the clamping force. The three arms are mutually matched to act when in work.

The driving arm component Q4400 includes an arm support Q4312, and the arm support Q4312 is a gantry frame. The lower part of the arm support Q4312 is connected with a base device. Two groups of Y-axis linear guide rails Q4308 are fixed on the top plane of the arm support Q4312 in parallel. A first sliding block slides on each Y-axis linear guide rail Q4308. The top surface of the first sliding block is fixed with a working plate Q4306. The top plane of the arm support Q4312 is arranged between the two Y-axis linear guide rails Q4308. Two groups of Y-axis screw rod motors Q4309 are arranged in parallel, and a screw rod of each group of Y-axis screw rod motors Q4309 is in matched transmission with a first threaded hole correspondingly arranged on the working plate Q4306. The first arm mechanism, the second arm mechanism and the third arm mechanism are sequentially arranged on the top surface of the working plate Q4306 in parallel in the direction close to the guide pipe bed. The Y-axis lead screw motor Q4309 is connected with a driving device on the base. Under the drive of a Y-axis lead screw motor Q4309, the working plate Q4306 can move left and right to complete the extending and retracting actions of the whole arm.

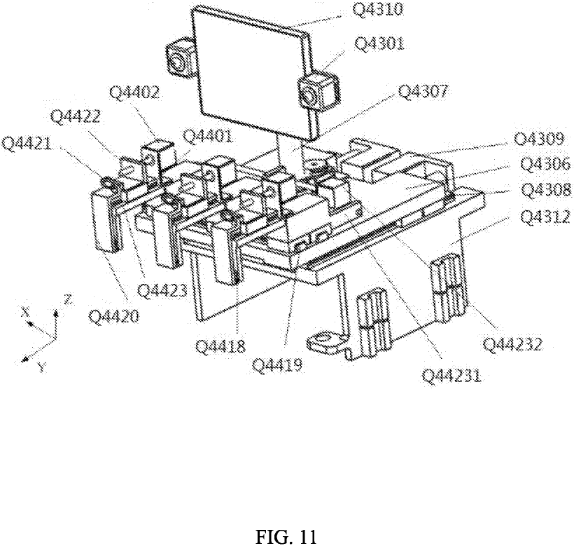

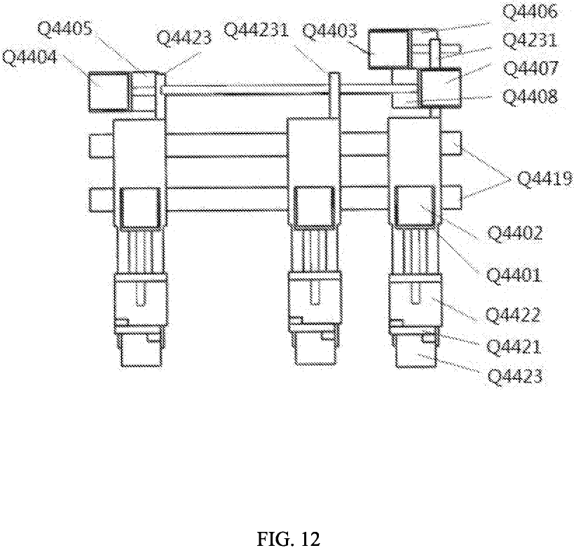

Referring to FIGS. 11 and 12, the first arm mechanism, the second arm mechanism and the third arm mechanism are of the same structure, and each of the first arm mechanism, the second arm mechanism and the third arm mechanism includes a right-angle frame Q4423. The right-angle frame Q4423 includes a connecting section and a clamping section which form an L shape. The connecting section slides on an X-axis linear guide rail Q4419 fixed on the arm support Q4312. A Y-direction guide rail is installed at the top of the connecting section. A third slide block slides on the Y-direction guide rail, a right-angle connecting piece Q4422 with a third threaded hole is fixedly connected to the top of the third slide block. The connecting section is located at the rear of the Y-direction guide rail. A Y-direction lead screw motor Q4402 is fixed through a Y-direction motor support Q4401, a lead screw of the Y-direction lead screw motor Q4402 is in fit transmission with the third threaded hole, and the Y-direction lead screw motor Q4402 is connected with a driving device. The front part of the right-angle connecting piece Q4422 is connected with a clamping piece Q4420, and the clamping piece Q4420 is matched with the clamping section to form the clamping mechanism. The Y-direction screw motor Q4402 drives the third slide block to move, so that the clamping of the clamping section and the clamping piece Q4420 is realized.

Advantageously, referring to the FIG. 11, the front end of the right-angle connecting piece Q4422 is provided with a first connecting boss. The clamping piece Q4420 is a right-angle piece. The upper part of the right-angle piece Q4422 is provided with a second connecting boss matched with the first connecting boss. The first connecting boss and the second connecting boss are connected. The clamping section is vertical to the connecting section and faces downwards, and is positioned at the front part of the working plate and the arm support and close to the side of the catheter bed. The shape of the lower part of the clamping piece Q4420 is the same as that of the clamping section. A clamping area is formed between the clamping piece Q4420 and the clamping section, and medical silica gel pieces Q4418 are fixed on the inner walls of two sides of the clamping area to prevent slipping.The first connecting boss and the second connecting boss are arranged in a staggered mode and used for being connected with two ends of a pressure sensor Q4421. The pressure sensor Q4421 is connected with a host of the base device and used for detecting the clamping force. The clamping force is thus fed back to the host machine through the pressure sensor Q4421, which sends commands to the control device to drive the corresponding motor. The clamping force is ensured to be controllable.

X-direction driving plates Q44231. are connected to the sides, away from the clamping section, of the corresponding right-angle frames Q4423 in the first arm mechanism, the second arm mechanism and the third arm mechanism. Each X-direction driving plate Q44231 corresponds to one group of X-direction driving mechanisms Q44232. The X-direction driving mechanisms Q44232 are connected with a driving device. The X-direction driving mechanism Q44232 is convenient to drive, three X-direction driving plates Q44231 are different in length according to arrangement, and three groups of driving screw motors (Q4404. Q4403 and Q4407) and three groups of driving motor supports (Q4405. Q4406 and Q4408) are correspondingly arranged.

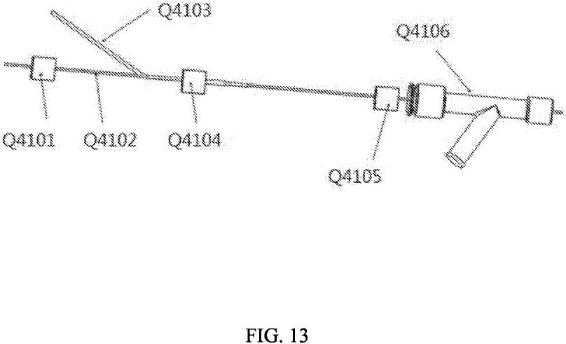

In FIGS. 12 and 13, the first arm mechanism, the second arm mechanism, and the third arm mechanism are arranged in this order from left to right. The arm component faces the guide tube side, and it is necessary to control the first arm mechanism by the robot to grip the guide wire at the tip Q4105 and then fix the first arm mechanism to be stationary. The third arm mechanism grasps the tip end of the catheter Q4103, the second arm mechanism grasps the tail end of the guide wire Q4102, adjusts the position to insert the catheter Q4103 into the guide wire Q4102, then the third arm mechanism pushes the catheter Q4103 forward, when approaching the second arm mechanism, the second arm mechanism is released. The second arm mechanism is then controlled to move about 2 cm in the Y valve direction. The second arm mechanism is controlled to clamp the guide wire. The third arm mechanism is controlled to continue to push the catheter Q4103 forward, and the process is sequentially repeated until the tail end of the guide wire Q4102 passes through the guide wire Q4103 by about 3 cm. The third arm mechanism is operated to retreat and moves to the rear end Q4101, and the guide wire Q4102 exposed from the middle end of the catheter Q4103 is clamped. Then, the second arm mechanism is controlled to push the guide wire Q4103 in the Y valve direction while holding the guide wire Q4103 at the middle end Q4104. The second arm mechanism pushes the guide wire Q4103 forward. The third arm mechanism is operated to move backward until the guide wire Q4102 is pulled into line. When the second arm mechanism moves to the first arm mechanism, the movement of the second arm mechanism is stopped, and the first arm mechanism is controlled to hold the Y valve Q4106. The second arm mechanism pushing guide wire Q4103 is controlled to enter the Y valve Q4106. After reaching the Y valve, the second arm mechanism is released and retreated by about 2 cm. The pinch guide wire is moved forward again, and the process is repeated until the head end portion of the guide wire Q4103 completely enters the Y valve Q4106.

When the catheter Q4103 needs to be removed from the guide wire Q4102, both the catheter and the guide wire are in the human blood vessel. During withdrawal of the catheter, it is necessary to ensure that the displacement of the guide wire does not change. The third arm mechanism is controlled to clamp the guide wire Q4102 which is 2-3 cm away from the outlet of the Y valve Q4106. The first arm mechanism is controlled to clamp the Y valve Q4106, and the second arm mechanism is controlled to clamp the guide pipe Q4103 at the outlet of the Y valve Q4106. ensuring the position of the third arm mechanism to be fixed. The second arm mechanism is controlled to move backwards until the second arm mechanism is close to the third arm mechanism and then stop, and then the third arm mechanism is released to move backwards for 2 cm to clamp the guide wire. The second arm mechanism is controlled to move backwards continuously until the second arm mechanism is close to the third arm mechanism and then stop. The steps is sequentially repeated until the head end of the catheter is 2-3 cm away from the tail end of the Y valve Q4106, and then the first arm mechanism is controlled to move to the position of the outlet of the Y valve Q4106. The guide wire is clamped and the guide wire is kept still. The second arm mechanism and the third arm mechanism is then controlled to move backwards together until the catheter leaves the guide wire. When the guide wire is replaced, the guide wire is firstly inserted into the Y valve Q4106, and the third arm mechanism and the second arm mechanism are controlled to clamp the position of the guide wire close to the head end. The first arm mechanism clamps the Y valve Q4106 and keeps the position of the Y valve Q4106 still. The third arm mechanism and the second arm mechanism are controlled to push the guide wire forward to enter the Y valve Q4106. The second arm mechanism is moved to the outlet of the Y valve and stops, then the third arm mechanism is released and moves backwards by 2 cm. The guide wire is then clamped, and the second arm mechanism is released and the guide wire is clamped after moving backwards by 2 cm. The third arm mechanism and the second arm mechanism move forward together until the second arm mechanism stops after moving to the outlet of the Y valve, and the cycle is stopped until 10-12 groups of actions are completed. When the guide wire Q4102 needs to be removed, the first arm mechanism is controlled to clamp the Y valve Q4106 and to keep the position thereof fixed. The third arm mechanism is controlled to move to the exit of the Y valve Q4106, grip the guidewire Q4102. and then move backwards until after the head end of the guidewire Q4102 is all clear of the tail end of the Y valve Q4106, at which time it is verified that the guidewire has been completely removed.

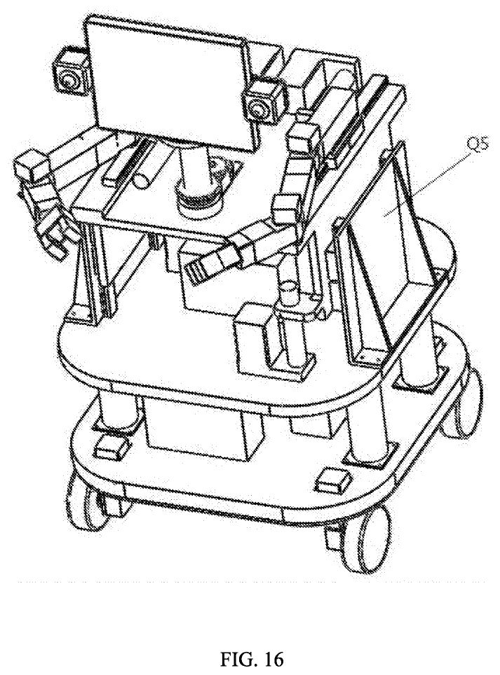

Consumable delivery robot Q5, see FIG. 16, is used for automatic management and delivery operation consumable. The full process is automated, the manpower resources are saved. The device overall is of compact structure, small, and is especially adapted for the environment of the catheter chamber. The consumable delivery robot includes a base device, a head and an executing device.

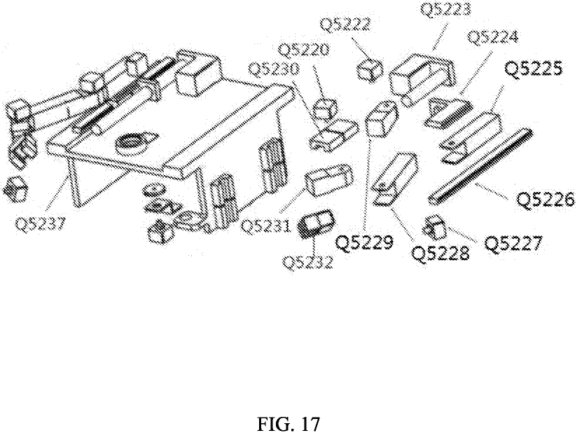

Referring to FIG. 17, the actuator includes two arm assemblies symmetrically disposed on both sides of the head. The arm component includes an arm linear guide rail Q5226 fixed on the top surface of the U-shaped lifting frame Q5237. An arm sliding block Q5230 is connected to the arm linear guide rail Q5226 in a sliding manner. A connecting piece Q5224 is fixed on the arm sliding block Q5230, and a rear arm Q5225 is fixed on the connecting piece Q5224. The front end of the rear arm Q5225 is rotatably connected with a middle arm connecting piece Q5229. A fifth servo motor Q5222 is arranged on the rear arm Q5225, and a power shaft of the fifth servo motor Q5222 is fixed to the middle arm connecting piece Q5229. The middle arm connecting piece Q5229 and the middle arm Q5228 are fixed. The front end of the middle arm Q5228 is rotatably connected with a front arm Q5231. A sixth servo motor Q5220 is mounted on the middle arm Q5228, and a power shaft of the sixth servo motor Q5220 is fixed with the front arm Q5231. A seventh servo motor Q5227 is fixed at the front end of the front arm Q5231, and a third electric hand grip Q5232 is fixed on a power shaft of the seventh servo motor Q5227. An arm lead screw motor Q5223 is mounted on the U-shaped lifting frame Q5237, and a shaft end lead screw of the arm lead screw motor Q5223 is in threaded connection with a threaded hole in the side face of the connecting piece Q5224.

Consumable delivery robot Q5 is used to transport surgical consumables to the interventional surgery. The base and lift portion mainly used to realize the removal and lift of robot. The base controls the movement of the whole robot and includes four groups of wheel devices controlled by motors. The lift portion can raise or lower the height of the head assembly and arm component of the system. The head and the actuator are mainly used for completing system identification and arm control. The head is the viewing and output end of the system. Two cameras are provided for observing external environment. The second touch screen is arranged for feeding back information to the user and receiving instructions of the user and is a control end of the user. The head can move in all directions to realize better observation of the environment. The arm component is used for finishing clamping the object. The arm component can stretch back and forth, and when the arm component needs to be grabbed, the arm can be unfolded. After the clamping is completed, the arm is retracted. Through the arm component, snatching of the operation consumptive material and the opening of the packing are achieved.

Advantageously, the consumable delivery robot Q5 for delivering consumptive material can be used with current consumptive material management system. Also it can be equipped with one set of consumptive material management system alone, and communicate wirelessly through bluetooth with consumptive material management system, thus the collaborative work is achieved.

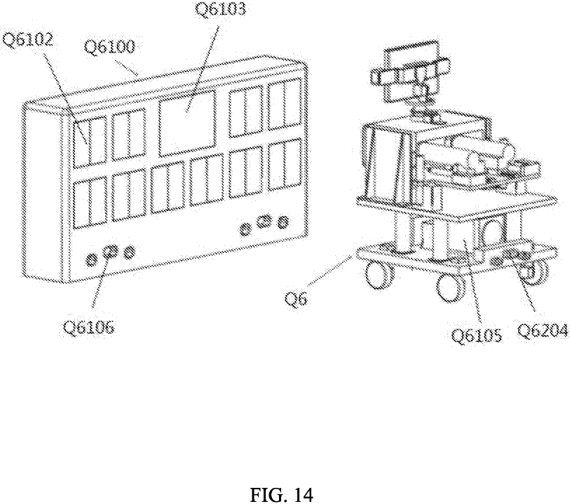

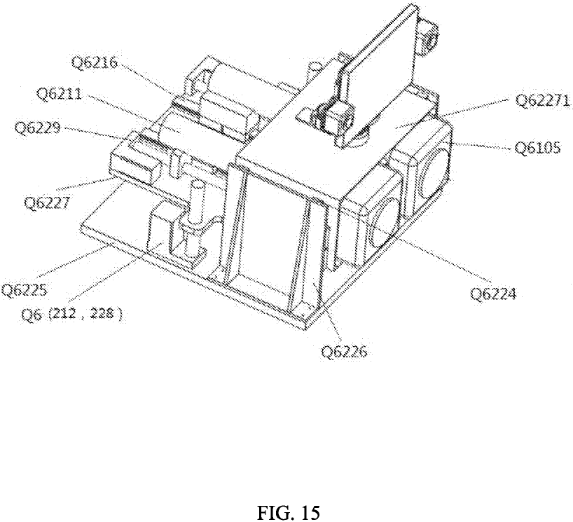

The fast charging robot Q6, see FIGS. 14 and 15, quickly charges each robot in the operating room, and ensures that each robot can continuously work. The fast charging robot Q6 is matched with a charging cabinet in a charging area for use, and the charging cabinet is used for charging the battery and the charging robot. The mode that the fast charging robot automatically replaces batteries for other robots is adopted. The whole battery replacing process is quick, and the battery replacing can be completed within one minute. After receiving the signal of needing to change the battery, the charging robot can automatically carry a fully charged battery, be driven to the side of the robot needing to change the battery, replace the original battery of the robot with the new one. The original battery is then brought back to the charging cabinet outside the operating room and charged. The whole process can be automatically completed by a robot Also included are a base unit, a head unit and a torso unit

The charging cabinet Q6100 is positioned outside an operating room (charging area 9). A human-computer interaction touch screen Q6103 is arranged on the charging cabinet Q6100. The controlling host is arranged in a shell of the charging cabinet Q6100 and is connected with a network where an interventional surgery robot is positioned. A signal of which specific robot needs to change a battery can be received, and the touch screen Q6103 receives and feeds back information and transmits the information to the controlling host for data processing and storage. A transformer for providing stable and proper power supply input is arranged in the shell. The external part of the surgical robot is provided with a plurality of charging grids Q6102 for charging, and ten charging grids can be arranged to meet the requirement of the surgical robot for charging the battery. Each charging grid Q6102 is provided with a charging socket matched with a plug of a battery Q6105. A robot charging hole is arranged below the charging grid Q6102. The fast charging robot Q6 is an automatic walking robot, and is in communication with the controlling host and used for replacing a battery for a robot with low electric quantity in an interventional surgery robot, putting the replaced battery into the charging grid opening Q6102 for charging, and is provided with a charging head Q6204 matched with the charging hole.

During charging, a charging hole at the bottom of the battery Q6105 is connected with a charging plug in the charging cabinet Q6100. An iron sheet is installed at the front end of the battery Q6105, and the iron sheet can be adsorbed and connected with an electromagnet in the charging cabinet, so that the battery is fixed. When a battery Q6105 is put into the charging cell Q6102, the system will start to automatically charge the battery until the battery is fully charged, and automatically cuts off the power supply and gives a prompt on the touch screen, and at the same time sends information to the charger robot Q6200, so that the robot can quickly find the ready battery. The system can support ten groups of batteries to be charged simultaneously, so that the battery replacement requirements of a plurality of robots can be effectively met. When the charging robot is dead, the charging robot can be charged on a charging platform below the charging cabinet. Two groups of charging station are set up on the charging cabinet, so that two fast charging robot Q6 can be charged simultaneously.

The trunk device is used for pushing and retracting the battery and lifting the system. The trunk device can stretch back and forth and stretch up and down, and the battery is pushed out when the battery needs to be pushed. After the action is finished, the operation can be retracted. The battery can be replaced by the overall cooperation of the system.

Referring to FIG. 15, specifically, the support plate Q6225 is included, the support plate Q6225 is located on the walking chassis of the base device, and the top of the support plate Q6225 is provided with a containing lattice Q62271 which can move relative to the vertical direction. The containing lattice Q62271 is provided with two horizontally arranged containing grids. One of which is used for containing a fully charged battery Q6105 for transportation, and the other is used for containing a battery Q6105 with low transportation electric quantity. The containing lattice Q62271 is connected with the head mechanism at the top part thereof. One end of the connecting plate Q6227, which is far away from the head mechanism, extends outwards to form a connecting plate Q6227. A clamping and pushing mechanism which can move telescopically, is arranged on the connecting plate Q6227 relative to the two accommodating grids, and the clamping and pushing mechanism is connected with a driver.

Two sides of the containing lattice Q62271 are fixed on the supporting plate Q6225. Two supports Q6226 are arranged on the two sides of the containing lattice Q62271. A vertical linear guide rail Q6224 is fixed on the inner side face of each support Q6226. A first sliding block is connected to each vertical linear guide rail Q6224 in a sliding mode, and the first sliding blocks on the two groups of supports Q6226 are fixed to the outer wall of the containing lattice Q62271. The two sides of the connecting plate Q6227 are respectively provided with a first threaded hole. Two vertical screw rod motor components Q6212 fixed on the supporting plate Q6225 are arranged. A screw rod in the Q6228 is matched with the two first threaded holes, and the vertical screw rod motor components Q6212 and Q6228 are electrically connected with a driver. Therefore, the lifting is completed through the cooperation of the vertical screw rod motor assemblies Q6212 and Q6228 and the first threaded holes on the left side and the right side of the connecting plate Q6227 respectively.

Referring to the FIG. 15, the clamping and pushing mechanism includes two groups of horizontal linear guide rails Q6216. The horizontal linear guide rails Q6216 are fixed on the connecting plate Q6227 and located behind the accommodating grid Q62271. A second slider is connected to each horizontal linear guide rail Q6216 in a sliding manner, a push rod Q6211 is fixed to each group of second sliders, a second threaded hole is formed in the bottom of each push rod Q6211, and an electromagnet is arranged at the end of each push rod Q6211 and used for adsorbing iron sheets on the battery Q6105. The connecting plate Q6227 is located outside the two groups of horizontal linear guide rails Q6216. and is fixed with two horizontal lead screw motor assemblies Q6229. a lead screw of the horizontal lead screw motor assembly Q6229 is matched with the second threaded hole, and the lead screw is electrically connected with the driver. Therefore, the horizontal lead screw motor assembly Q6229 is matched with the second threaded hole, the push rod is extended out and retracted, and the electromagnet at the front end of the push rod is matched with an iron sheet on the battery and used for clamping the battery.

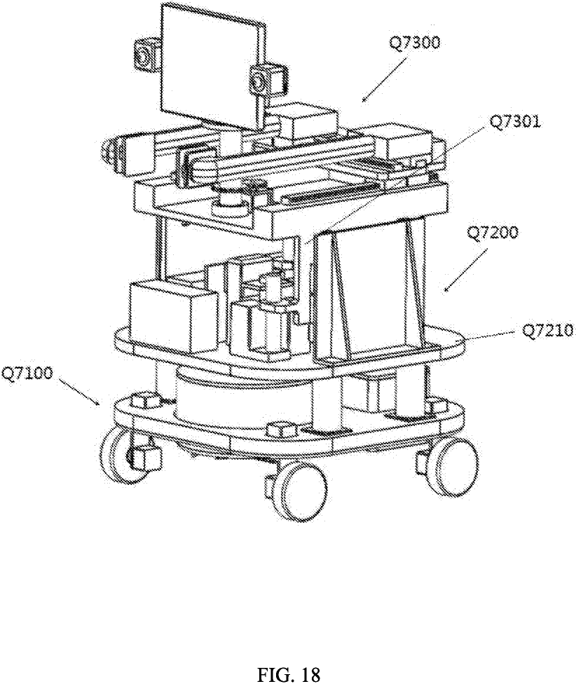

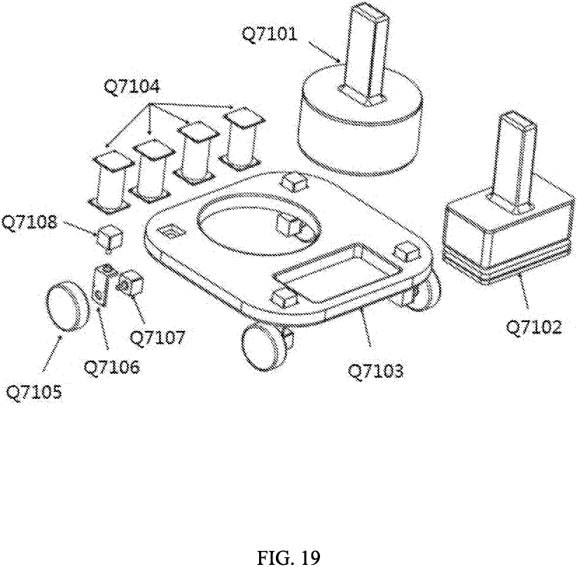

The cleaning robot Q7, see FIGS. 18 and 19, mainly includes three parts, that is, a base device Q7100, a trunk device Q7200, and a head and arm component Q7300. The whole device can move in the interventional operation catheter chamber, the cleaning of the ground in the catheter chamber, the catheter bed and other objects is completed, and the clean and sterile environment of the catheter chamber is ensured.

The base device Q7100 is mainly used for realizing the movement of the robot and finishing the ground cleaning action. The base device Q7100 includes a movement device, a sweeping device Q7101 and a floor wiping device Q7102. The movement device is used for controlling the movement of the whole robot. The sweeping device Q7101 is used for finishing the sweeping action of the robot and sweeping the garbage and dust on the ground, and the floor wiping device Q7102 is used for finishing the floor wiping action of the robot. The movement device, the floor sweeping device Q7101 and the floor wiping device Q7102 are integrally installed on a bottom plate Q7103, and vertical columns Q7104 for supporting the trunk device Q7200 are respectively installed at four corners of the upper surface of the bottom plate Q7103.

The movement device includes four sets of wheel assemblies which are of the same structure and are driven by a motor. One set of the wheel assemblies is explained below. The wheel assembly includes a wheel Q7105, a wheel connecting plate Q7106, a first servo motor Q7107 and a second servo motor Q7108. The wheel connecting plate Q7106 is right-angled, and a wheel connecting plate through hole is formed in a vertical plate at the lower part of the wheel connecting plate Q7106. The first servo motor Q7107 is fixed in the inner space of the wheel connecting plate Q7106. A motor shaft of the first servo motor Q7107 penetrates through a through hole of the wheel connecting plate to be connected with a wheel Q7105 on the outer side of a lower vertical plate, and the first servo motor Q7107 controls the forward and backward movement of the wheel Q7105. A motor mounting hole is respectively formed in each of four corners of the bottom plate Q7103. A second servo motor Q7108 is fixed in the motor mounting hole in the corresponding position on the bottom plate Q7103. A motor shaft of the second servo motor Q7108 penetrates through the motor mounting hole to be connected with an upper transverse plate of the wheel connecting plate Q7106, and the second servo motor Q7108 controls the steering of the wheel Q7105. The first servo motor Q7107 is matched with the second servo motor Q7108, and the four wheels Q7105 move together, so that the all-directional movement of the whole robot can be realized.

The trunk device Q7200 is mainly used for supporting and lifting the robot and includes a supporting plate Q7210, and a control and power supply device, a floor sweeping control device, a floor wiping control device and a lifting device which are arranged on the supporting plate Q7210. The bottom of the supporting plate Q7210 is connected with the top of the upright post Q7104. The control and power supply equipment supplies power to the system and is used as a control information processing center of the whole system. The sweeping control device is connected with the sweeping device Q7101 and used for controlling the height of the sweeping device Q7101 and realizing different working modes of the system. The floor wiping control device is connected with the floor wiping device Q7102 and used for controlling the height of the floor wiping device Q7102 and realizing different working modes of the system. The lifting device is connected with the head and arm component Q7300 and is used for lifting or lowering the height of the head and arm component Q7300.

The head and arm component Q7300 is mainly used for completing system identification and arm control, and includes a head and arm component support Q7301, a head device and an arm component. The head device and the arm component are arranged on the head and arm component support Q7301, the head and arm component support Q7301 is connected with a lifting device. The head device is an observation and output end of the system, and the aim component is used for completing clamping of an article. The head unit can be moved in all directions to achieve a better view of the environment. The arm component is used for finishing clamping the objects. The arm component can be stretched back and forth, and when the arm component needs to be grabbed, the arm is unfolded. After clamping, the arm is retracted. Through the arm system, the cleaning of the articles on the catheter bed can be completed.

The base device and the head device of each robot can be of the same structure, so that the cost is reduced, and the interchangeability of robot parts is improved. The base can realize automatic walking, and 360 degrees rotations of level, and every single move can be realized to head or head device, and convenient nimble observation surrounding environment Each robot is provided with a communication module, so that the communication performance of the whole system is realized.

As for the contrast agent injection device, an electric contrast agent injector disclosed in patent CN215608391U can be used.

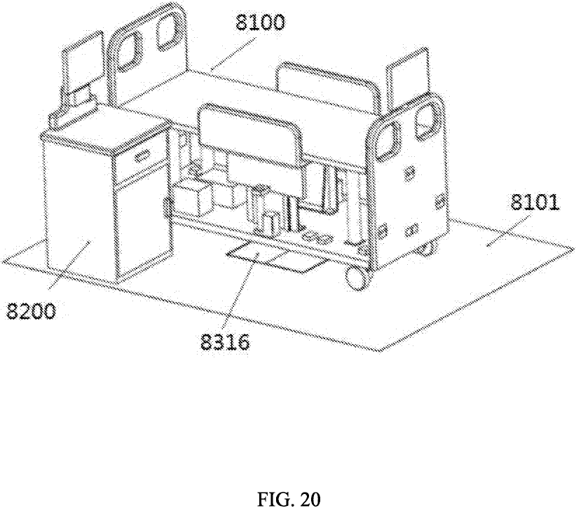

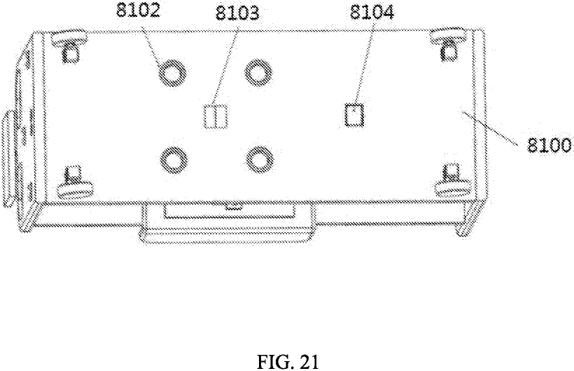

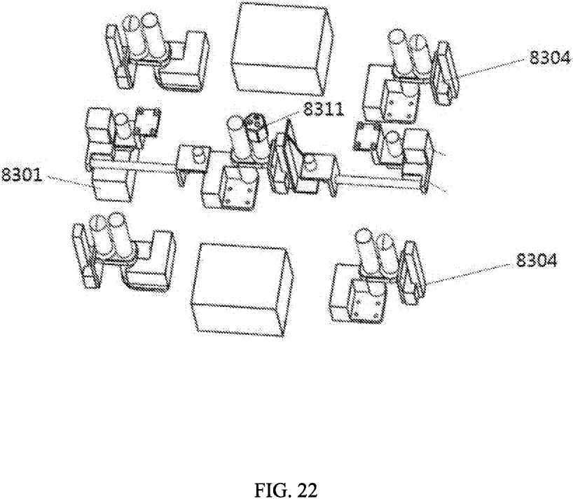

The automatic transfer trolley 8, which may be an existing transfer trolley, may also be a transfer system with an automatic charging function, see FIGS. 20-22. The automatic transfer trolley 8 includes a transfer trolley 8100, an automatic fixing and automatic charging device (corresponding to the transfer trolley charging post 81). The automatic fixing and automatic charging device includes a shell, a fixing mechanism 8304, an automatic charging mechanism and a control cabinet 8200. The shell is hidden and is embedded in the underground 8101. A cover 8316 of the shell can be opened to expose an accommodating cavity formed in the shell. The automatic charging mechanism and the fixing mechanism 8304 is positioned in the accommodating cavity and used for automatically extending out and fixing the transfer trolley. The automatic charging mechanism is positioned in the accommodating cavity, and a charging plug 8311 of the automatic charging mechanism is connected with an external power supply, can stretch into the accommodating cavity and is used for charging the transfer trolley. The control cabinet 8200 is used for displaying, storing information, setting parameters and processing data, and is connected with the automatic charging mechanism and the fixing mechanism 8304. The scheme can guarantee that automatic transfer device charges, and then guarantee that automatic transfer device works normally.

The automatic transfer device has a fixing portion at the bottom thereof to be fitted with the automatic charging mechanism and the fixing mechanism 8304, and a charging head at the bottom thereof to be fitted with the charging plug 8311. The two poles of the charging plug 8311 are respectively connected with the live wire and the zero line of the network power supply, and are also provided with a travel switch for detecting whether the two plugs are connected.

Specifically, the bottom of the automatic transfer device is provided with four fixing holes 8102 corresponding to the four fixing mechanisms 8304. The charging head 8110 is located in flip 8103 of automatic transfer device bottom. The flip 8103 opens downwards, and position sensor 8104 is installed to its next door. The position sensor 8104 communicates with position sensor basic station 8301. After the fixing rods of the fixing mechanisms 8304 are inserted into the fixing holes, the automatic transfer device is firmly fixed, so that the vehicle body cannot move randomly.

Position sensor 8104 is used for cooperating with position sensor basic station 8301, and supplement a positioning of the automatic transfer device. After the automatic transfer device receives the coordinates of the position sensor base station 8301, the movable vehicle body is controlled to move, so that the automatic transfer device reaches the determined position.

The DSA apparatus may be implemented using existing technologies.

In the description herein, references to the description of the term “one embodiment,” “some embodiments,” “an example,” “a specific example.” or “some examples,” etc., mean that a particular feature, structure, material, or characteristic described in connection with the embodiment or example is included in at least one embodiment or example of the invention. In this specification, the schematic representations of the terms used above are not necessarily intended to refer to the same embodiment or example. Furthermore, the particular features, structures, materials, or characteristics described may be combined in any suitable manner in any one or more embodiments or examples. Furthermore, various embodiments or examples described in this specification can be combined and combined by those skilled in the art. Although embodiments of the disclosure have been shown and described above, it is understood that the above embodiments are exemplary and should not be construed as limiting the disclosure, and that variations, modifications, substitutions and alterations can be made to the above embodiments by those of ordinary skill in the art within the scope of the disclosure.

What is claimed is:

1. An interventional unmanned operation chamber system, comprising:
a catheter chamber, the catheter chamber being the area of an interventional surgery and having a catheter bed therein;
a control chamber, wherein the control chamber is arranged close to the catheter chamber, and an observation window is arranged between the catheter chamber and the control chamber;
a plurality of robots, wherein an interventional surgery robot a master control robot, a puncture robot and a catheter and guidewire replacing robot of the plurality of robots are matched with each other are arranged in the catheter chamber; the catheter bed is provided with a DSA device and a contrast agent injection device;
wherein the monitoring device is arranged in the control chamber and is in communication with the robot; the DSA device and the contrast agent injection device, and the monitoring device is configured for displaying information of each device and the robot, updating in real time and synchronously and supervision of doctors; and
the controller is arranged in the control chamber and is used for man-machine interaction between a doctor and the robot.

2. The interventional unmanned operation chamber system according to claim 1, wherein a ward is provided adjacent the catheter chamber for resting of the patient.

3. The interventional unmanned operation chamber system according to claim 2, wherein an automatic transfer trolley is movable between the ward and the catheter chamber for automated transfer of the patient.

4. The interventional unmanned operation chamber system according to claim 3, wherein a transfer trolley charging post is fixed in the ward for charging the automatic transfer trolley.

5. The interventional unmanned operation chamber system according to claim 1, wherein the robot further comprises a consumable delivery robot; the consumable delivery robot records surgical consumable information for delivering surgical consumables to the catheter chamber, which is communicatively connected with the monitoring device and the controller.

6. The interventional unmanned operation chamber system of claim 1, wherein the robot further comprises a fast charging robot, the fast charging robot being communicatively coupled to the plurality of robots for replacing low battery batteries.

7. The interventional unmanned operation chamber system according to claim 6, wherein a charging area for charging a low-battery is provided outside the catheter chamber, and a charging position for the fast charging robot is provided in the charging area.

8. The interventional unmanned operation chamber system according to claim 1, further comprising a cleaning robot in communication with the controller, and the cleaning robot being configured to automatically clean the catheter chamber after the procedure is completed.

9. The interventional unmanned operation chamber system according to claim 1, wherein the monitoring device comprises a plurality of display screens supported by screen supports.

10. The interventional unmanned operation chamber system according to claim 1, wherein the authority of the robot has priority and the master control robot has the largest authority, being the director of the operation, for image diagnosis, instructing other robots to work in concert.
